(19) United States
(12) Patent Application Publication
(10) Pub. No.: US 2008/0227747 A1
(43) Pub. Date: Sep. 18, 2008

(54) COMPOSITION AND METHODS FOR TREATING OR PREVENTING DEGENERATIVE JOINT AND CARDIOVASCULAR CONDITIONS

(76) Inventor: Philip TABBINER, Wilmington, NC (US)

Correspondence Address: BRINKS, HOFER, GILSON & LIONE, 2801 SLATER ROAD, SUITE 120, MORRISVILLE, NC, 27560, US

(21) Appl. No.: 11/686,734

(22) Filed: Mar. 15, 2007

(51) Int. Cl. A61K 31/726; A61K 31/045; A61K 31/5025; A61P 9/00; A61P 19/00; A61K 31/70

(52) U.S. Cl. 514/54; 514/249; 514/62; 514/724

(57) ABSTRACT

A therapeutic composition and methods for the treatment and prevention of a degenerative joint disorder and/or a cardiovascular disease comprising polycosanols, glucosamine and chondroitin are disclosed.

### FIELD OF THE INVENTION

The subject of this invention relates to compositions and methods of treatment and prevention of degenerative and/or cardiovascular conditions.

### BACKGROUND OF THE INVENTION

Degenerative joint disorders is a painful degenerative condition that results in the detcrioration of cartilage tissues that support the weight-bearing joints in the body. Once the cartilage is thinned or lost, the constant grinding of bones against each other causes pain and stiffness around the joint. Abnormal and excess bone formations called spurs grow from the damaged bones, causing further pain and stiffness. It is believed that degenerative joint disorders affect 80% of people over the age of 60. Degenerative joint disorders include, for example, osteoarthritis, rheumatoid arthritis, other rheumatic disorders with cartilage breakdown, chondrolysis after joint trauma, for example, after meniscus or patella injuries or torn ligaments, or chondrolysis associated with prolonged immobilization of joints. Arthritis is a general term for over 100 conditions that affect the joints and surrounding tissues. The two most common types of arthritis are osteoarthritis and rheumatoid arthritis.

Osteoarthritis is a painful, degenerative joint disease that often involves the hips, knees, neck, lower back, or the small joints of the hands. Osteoarthritis usually develops in joints that are injured by repeated overuse in the performance of a particular job or a favorite sport or from carrying around excess body weight. Eventually this injury or repeated impact thins or wears away the cartilage that cushions the ends of the bones in the joint so that the bones rub together, causing a grating sensation. Joint flexibility is reduced, bony spurs develop, and the joint swells. Usually, the first symptom a person has with osteoarthritis is pain that worsens following exercise or immobility.

Rheumatoid arthritis is an autoimmune inflammatory disease in which the body releases enzymes that attack its own healthy tissues. In rheumatoid arthritis, these enzymes destroy the linings of joints causing pain, swelling, stiffness, deformity, and reduced movement and function. Rheumatoid arthritis also may include systemic symptoms. There is no cure for osteoarthritis or rheumatoid arthritis, however, several drugs and medication options are approved for the prevention and treatment of these conditions.

Cardiovascular diseases and cancer are the most common natural causes of death. The cardiovascular diseases include many serious diseases that involve the cardiac and vascular systems, such as atherosclerosis, ischemic heart diseases, cardiac failure, cardiac shock, arrhythmia, hypertension, cerebral vascular diseases and peripheral vascular diseases. Atherosclerosis most often occurs as a complication of hyperlipidemia and may be treated with antihyperlipidemic agents. Ischemic heart disease, cardiac failure, cardiac shock, cerebral vascular disease, peripheral vascular disease, hypertension, arrhythmia and arteriosclerosis may be fatal because ischemia develops in various organs such as the heart, brain and the walls of blood vessels. The ischemia damages the organs in which it develops because it impairs the functions of mitochondria that produce adenosine triphosphate (ATP). The resulting functional damage of organs can be fatal if it occurs in vital organs such as the heart, brain and blood vessels. Antiarrhythmic agents have been used to treat ischemic heart disease and arrhythmia, but their use with patients with possible cardiac failure has been limited because these agents may cause cardiac arrest by their cardiodepressant effects.

The cardiovascular diseases named above may develop independently, but more often than not, they occur in various combinations. For example, ischemic heart diseases are frequently accompanied by arrhythmia and cardiac failure, and complications of cerebrovascular disorder with hypertension are well known. Atherosclerosis is often complicated by one or more cardiovascular diseases and can make the patient seriously ill. For some subjects, degenerative joint disorder may co-exist with cardiovascular disease.

### SUMMARY

The present invention is useful to treat, reduce, or prevent either one or both of degenerative joint disorders and cardiovascular disease by administering to a subject in need thereof a therapeutically effective amount of a composition comprising polycosanols, glucosamine and chondroitin, which may be adapted for oral administration. Methods and compositions for the treatment and prevention of degenerative joint disorders and cardiovascular disease are provided.

In embodiments, a therapeutic composition is provided comprising polycosanols, chondroitin, glucosamine and optionally para aminobenzoic acid (PABA), folic acid or combinations thereof. The polycosanols comprise at least two fatty alcohols selected from tetracosanol, hexacosanol, octacosanol and triacontanol.

In embodiments, a composition for treating and/or reducing a risk of a cardiovascular disease in a subject in need thereof is provided. The composition comprises polycosanols, at least about 10 milligrams of chondroitin per milligram of the polycosanols, at least about 10 milligrams of glucosamine per milligram polycosanols, optionally at least about 10 milligrams of PABA per milligram of the polycosanols and optionally at least about 0.005 milligrams of folic acid per milligram polycosanols.

In embodiments, a method for treating and/or reducing a risk of a cardiovascular disease in a subject in need thereof is provided. The method comprises providing a formulation comprising polycosanols, at least about 10 milligrams of chondroitin per milligram of the polycosanols, at least about 10 milligrams of glucosamine per milligram polycosanols, optionally at least about 10 milligrams of PABA per milligram of the polycosanols and optionally at least about 0.005 milligrams of folic acid per milligram polycosanols. The method provides for orally administering to the subject for a therapeutically effective period of time a therapeutically effective amount of the formulation.

In embodiments, a composition for treating and/or reducing a risk of a degenerative joint disorder in a subject in need thereof is provided. The composition comprises polycosanols, at least about 10 milligrams of chondroitin per milligram of the polycosanols, at least about 10 milligrams of glucosamine per milligram polycosanols, optionally at least about 10 milligrams of PABA per milligram of the polycosanols and optionally at least about 0.005 milligrams of folic acid per milligram polycosanols. The polycosanols comprise at least two fatty alcohols selected from tetracosanol, hexacosanol, octacosanol and triacontanol.

In embodiments, a method for treating and/or reducing a risk of a degenerative joint disorder in a subject in need thereof is provided. The method comprises providing a formulation comprising polycosanols, at least about 10 milligrams of chondroitin per milligram of the polycosanols, at least about 10 milligrams of glucosamine per milligram polycosanols, optionally at least about 10 milligrams of PABA per milligram of the polycosanols and optionally at least about 0.005 milligrams of folic acid per milligram polycosanols. The polycosanols comprise at least two fatty alcohols selected from tetracosanol, hexacosanol, octacosanol and triacontanol. The method provides for orally administering to the subject for a therapeutically effective period of time a therapeutically effective amount of the formulation.

### DETAILED DESCRIPTION

In accordance with the embodiments herein disclosed, compositions and methods of treating and/or preventing degenerative joint disorders and/or cardiovascular diseases are provided. The methods and compositions of the present invention provide for the prevention or treatment of degenerative joint disorders and/or cardiovascular diseases.

As used herein, the terms “degenerative joint disorder” and “arthritic condition” are used interchangeably and refer to any etiological or pathological symptom affecting a joint. Such symptoms and etiology include swelling, pain and/or stiffness of a joint such that complete or partial loss of function and/or damage to the joint and/or a reduction of the joint mobility. The term also includes chronic inflammation, primarily of the synovial tissue, pannus formation, destruction of articular cartilage and release of various enzymes, for example, collagenase and lysosomal enzymes, in an affected joint, osteoarthritis, rheumatic disorders with cartilage breakdown, rheumatoid arthritis, chondrolysis after joint trauma, for example, after meniscus or patella injuries or torn ligaments, or chondrolysis associated with prolonged immobilization of joints. By way of example, degenerative joint disorder includes osteoarthritis, the rheumatic diseases, particularly rheumatoid arthritis, juvenile rheumatoid arthritis and psoriatic arthritis.

As used herein, the term “cardiovascular disease” refers generally to any etiological or pathological symptom affecting the cardiovascular system. Cardiovascular conditions includes, for example, cardiac arrhythmias, congestive heart failure, and stroke; reducing the incidence of cardiovascular disease-related events; preventing or treating vascular conditions and associated thrombotic events (e.g., atherosclerosis): preventing or treating vascular inflammation; methods for preventing and/or treating a disorder of lipid metabolism, including hyperlipidemia, sitosterolemia and arteriosclerotic symptoms; reducing the incidence of cardiovascular disease-related events; and/or treating or preventing vascular inflammation.

As used herein, “pharmacologically active agent” or “active agent” or “agent” are used interchangeably and refer to a compound or composition of matter which, when administered to a human or animal induces a desired pharmacologic and/or physiologic effect by local and/or systemic action.

As used herein, “subject in need thereof” refers to a mammal (e.g., a human, dog, horse, or cat).

As used herein, the term “composition” refers to mixtures of active ingredients, such as polycosanols, chondroitin and glucosamine, optionally together with suitable carriers and excipients in a desired arithmetically determined ratio, and which are obtained by conventional pharmaceutical methods.

The terms “pharmaceutical formulation” and “formulation” are used interchangeably and refer to any form of the composition commonly used for pharmaceutical administration, including solids, liquids, suspensions, oral strips, and gels. The term is also intended to encompass excipients, carriers, diluents, glidants and the like. The polycosanols, glucosamine and chondroitin composition may be all or part of a formulation adapted for a particular mode of administration. The formulation preferably is a pharmaceutically acceptable formulation.

As used herein, the term “administration” and its grammatical equivalents, when referring to use of a composition or formulation described herein, include, by way of example, administration of each agent in a substantially simultaneous manner in a regimen that will provide beneficial effects of the drug combination, and includes co-administration of these agents in a sequential mamler. Thus, for example, the polycosanol and the combination of chondroitin and glucosamine may be administered in one or more therapeutic dosage forms, such as one or more tablets, one or more capsules or one or more gel-caps. Combinations of tablets and/or capsules and/or gelcaps are envisaged. The compositions herein disclosed may be sequentially administered. Sequential administration includes, for example, both relatively short and relatively long periods between the administrations of each of the compounds of the present composition. Preferably, the compounds of the composition are administered while at least one compound is still having an efficacious effect on the subject. Administration of each compound in a substantially simultaneous manner is generally preferred. The simultaneous presence of polycosanol and the combination of chondroitin and glucosamine in a subject typically has a greater efficacy than the administration of any of said compound alone.

When the compounds are administered sequentially, it is preferred that the combination of agents are given to the subject within the therapeutic response time of at least one compound to be administered. For example, embodiments include the administration of polycosanol and the combination of chondroitin and glucosamine to the subject and the later administration of PABA. Preferably the PABA is administered to the subject while the polycosanol and the combination of chondroitin and glucosamine are still present in the subject at a level that is therapeutically effective.

The term “polycosanols” refers generally to one or more fatty alcohols extracted and/or derived from the waxes of plants such as sugar cane, rice bran and yams, as well as beeswax and Ericerits Pela wax secreations. Polycosanols as used herein may comprise one or more, preferably two or more, more preferably three or more of the fatty alcohols octanosol, triacontanol (melissyl alcohol or myricyl alcohol), hexacosanol, 1-heptacosanol, 1-nonacosanol, 1-dotriacontanol, and geddyl alcohol. Polycosanols includes extracts that may be subsequently treated such as by saponification or hydrolysis and then fractionated to produce a complex mixture that may be artificially enriched with a mixture of higher primary aliphatic alcohols.

The term “chondroitin” refers generally to sulfated glycosaminoglycan (GAG) comprising a chain of alternating sugars (N-acetylgalactosamine and glucuronic acid). For example, chondroitin may include chondroitin sulfate A, which is predominantly sulfated at carbon 4 of the N-acetylgalactosamine (GalNAc) sugar (chondroitin-4-sulfate), chondroitin sulfate B (dermatan sulfate), chondroitin sulfate C, which is predominantly sulfated at carbon 6 of the GalNAc sugar (chondroitin-6-sulfate), chondroitin sulfate D, which is predominantly sulfated at carbon 2 of the glucuronic acid and 6 of the GalNAc sugar (chondroitin-2,6-sulfate), chondroitin sulfate E, which is predominantly sulfated at carbons 4 and 6 of the GalNAc sugar (chondroitin-4,6-sulfate), salts and mixtures thereof. By way of example, chondroitin is chondroitin sulfate sodium.

Chondroitin may obtained from any natural or synthetic source. Chondroitin may be obtained from extracts of cartilaginous shark, fish and bird cartilage or cow or pig tissue, such as the trachea, ear and nose. As chondroitin may be obtained from a natural source and is naturally present in a wide variety of forms, the composition may vary. Chondroitin used herein may be as at least 50% bioequivalent to that of United States Pharmacopoeia (USP) identification and quantification testing standard, catalog No. 1133570, for chondroitin sulfate sodium. The dosage of chondroitin may be 500-2,500 mg per day. Higher or lower amounts of chondroitin may be administered.

The term “glucosamine” refers generally to an amino sugar precursor for the biochemical synthesis of glycosylated proteins and lipids. Glucosamine includes D-glucosamine (glucosamine-6-phosphate) which is synthesized from fructose-6-phosphate and glutamine. Glucosamine salt includes, for example, glucosamine sulfate and glucosamine hydrochloride. Glucosamine may be administered as a glucosamine salt in dosages of about 500-2500 mg per day. Higher or lower amounts of glucosamine may be administered. Glucosamine and chondroitin sulfate may further be combined with methylsulfonylmethane.

In embodiments, a therapeutic composition is provided. The therapeutic composition comprises polycosanols, chondroitin and glucosamine. The therapeutic composition may optionally comprise PABA, folic acid or combinations thereof. In embodiments, the polycosanols comprise at least two fatty alcohols selected from tetracosanol, hexacosanol, octacosanol and triacontanol.

In embodiments, the therapeutic composition may comprise an amount of polycosanols between 1 milligram and 100 milligrams. The therapeutic composition may comprise an amount of chondroitin between 10 milligrams and 2500 milligrams. The therapeutic composition may comprise an amount of glucosamine between 10 milligrams and 2500 milligrams. The therapeutic composition may comprise an amount of PABA between 10 milligrams and 2500 milligrams. The therapeutic composition may comprise an amount of folic acid between 0.005 milligrams and 5 milligrams.

In embodiments, the therapeutic composition comprises an amount of polycosanols between 10 milligram and 50 milligrams, an amount of chondroitin between 500 milligrams and 2500 milligrams and an amount of glucosamine between 500 milligrams and 2500 milligrams.

In embodiments, the therapeutic composition comprises an amount of polycosanols between 10 milligram and 50 milligrams, an amount of chondroitin between 500 milligrams and 2500 milligrams, an amount of glucosamine between 500 milligrams and 2500 milligrams and an amount of PABA between 500 milligrams and 2500 milligrams.

In embodiments, the therapeutic composition comprises an amount of polycosanols between 10 milligram and 50 milligrams, an amount of chondroitin between 500 milligrams and 2500 milligrams, an amount of glucosamine between 500 milligrams and 2500 milligrams, an amount of PABA between 500 milligrams and 2500 milligrams and an amount of folic acid between 0.01 milligrams and 1 milligram.

The therapeutic composition may further comprise an anti-inflammatory agent. The anti-inflammatory agent may be a NSAID. The therapeutic composition may further comprise a carrier for oral or buccal administration. The carrier may be any pharmaceutically acceptable carrier. The carrier may provide controlled-release and/or extended release profiles independently for one or more compounds of the composition.

In embodiments, a therapeutic composition is provided. The therapeutic composition consists essentially of polycosanols, at least about 10 milligrams of chondroitin per milligram of the polycosanols and at least about 10 milligrams of glucosamine per milligram polycosanols. In embodiments, the therapeutic composition further comprises at least about 10 milligrams of PABA per milligram of the polycosanols and/or at least about 0.005 milligrams of folic acid per milligram polycosanols. The therapeutic composition may comprise an amount of polycosanols between 10 milligram and 50 milligrams. The therapeutic composition may consist essentially of polycosanols and an amount of chondroitin between 500 milligrams and 2500 milligrams and an amount of glucosamine between 500 milligrams and 2500 milligrams. The therapeutic composition may consist essentially of polycosanols and an amount of chondroitin between 500 milligrams and 2500 milligrams, an amount of glucosamine between 500 milligrams and 2500 milligrams and an amount of PABA between 500 milligrams and 2500 milligrams and/or an amount of folic acid between 0.01 milligrams and 1 milligram.

In embodiments, a composition for treating and/or reducing a risk of a degenerative joint disorder in a subject in need thereof is provided. The composition comprises polycosanols, at least about 10 milligrams of chondroitin per milligram of the polycosanols and at least about 10 milligrams of glucosamine per milligram polycosanols. The polycosanols comprise at least two fatty alcohols selected from tetracosanol, hexacosanol, octacosanol and triacontanol. In embodiments, the formulation includes at least about 10 milligrams of PABA per milligram of the polycosanols. In embodiments, the formulation includes at least about 0.005 milligrams of folic acid per milligram polycosanols. In embodiments, the formulation includes at least about 10 milligrams of PABA per milligram of the polycosanols and at least about 0.005 milligrams of folic acid per milligram polycosanols.

In embodiments, a composition for treating and/or reducing a risk of a cardiovascular disease in a subject in need thereof is provided. The composition comprises polycosanols, at least about 10 milligrams of chondroitin per milligram of the polycosanols and at least about 10 milligrams of glucosamine per milligram polycosanols. The polycosanols comprise at least two fatty alcohols selected from tetracosanol, hexacosanol, octacosanol and triacontanol. In embodiments, the formulation includes at least about 10 milligrams of PABA per milligram of the polycosanols. In embodiments, the formulation includes at least about 0.005 milligrams of folic acid per milligram polycosanols. In embodiments, the formulation includes at least about 10 milligrams of PABA per milligram of the polycosanols and at least about 0.005 milligrams of folic acid per milligram polycosanols.

In embodiments, methods are provided comprising administering a composition or formulation described herein. The methods comprise administering a therapeutically effective amount of a composition or formulation described herein to a patient at risk of or in need of treatment of degenerative joint disorders and/or cardiovascular diseases. The method also includes co-administering a therapeutically effective amount of a compound or formulation described herein and at least one other additional therapeutic agent.

In embodiments, a method for treating and/or reducing a risk of a degenerative joint disorder in a subject in need thereof is provided. The method comprises providing a formulation comprising polycosanols, at least about 10 milligrams of chondroitin per milligram of the polycosanols and at least about 10 milligrams of glucosamine per milligram polycosanols and orally administering to the subject for a therapeutically effective period of time a therapeutically effective amount of the formulation. The polycosanols comprise at least two fatty alcohols selected from tetracosanol, hexacosanol, octacosanol and triacontanol. In embodiments, the formulation of the method includes at least about 10 milligrams of PABA per milligram of the polycosanols. In embodiments, the formulation of the method includes at least about 0.005 milligrams of folic acid per milligram polycosanols. In embodiments, the formulation of the method includes at least about 10 milligrams of PABA per milligram of the polycosanols and at least about 0.005 milligrams of folic acid per milligram polycosanols.

In embodiments, a method for treating and/or reducing a risk of a cardiovascular disease is provided. The method comprises providing a formulation comprising polycosanols, at least about 10 milligrams of chondroitin per milligram of the polycosanols and at least about 10 milligrams of glucosamine per milligram polycosanols and orally administering to the subject for a therapeutically effective period of time a therapeutically effective amount of the formulation. In embodiments, the formulation of the method includes at least about 10 milligrams of PABA per milligram of the polycosanols. In embodiments, the formulation of the method includes at least about 0.005 milligrams of folic acid per milligram polycosanols. In embodiments, the formulation of the method includes at least about 10 milligrams of PABA per milligram of the polycosanols and at least about 0.005 milligrams of folic acid per milligram polycosanois.

In embodiments, a method for treating and/or reducing a risk of a degenerative joint disorder and a cardiovascular disease in a subject in need thereof is provided. The method comprises providing a formulation comprising polycosanols and at least about 10 milligrams of chondroitin per milligram of the polycosanols and at least about 10 milligrams of glucosamine per milligram polycosanols and orally administering to the subject for a therapeutically effective period of time a therapeutically effective amount of the formulation. The polycosanols comprise at least two fatty alcohols selected from tetracosanol, hexacosanol, octacosanol and triacontanol. In embodiments, the formulation of the method includes at least about 10 milligrams of PABA per milligram of the polycosanols. In embodiments, the formulation of the method includes at least about 0.005 milligrams of folic acid per milligram polycosanols. In embodiments, the formulation of the method includes at least about 10 milligrams of PABA per milligram of the polycosanols and at least about 0.005 milligrams of folic acid per milligram polycosanols.

In embodiments, a method for preventing degenerative joint disorders and/or cardiovascular diseases in a subject, the method comprising administering to the subject polycosanols, glucosamine and chondroitin.

As used herein, the term “prevent” and its grammatical equivalents refer to any reduction of a subject's predisposition or risk for developing a degenerative joint disorders and/or cardiovascular diseases disorder or a degenerative joint disorder- and cardiovascular disease-related complication. The term “prevent” includes either preventing a clinically evident degenerative joint disorder and/or cardiovascular disease from occurring altogether or preventing a preclinically evident degenerative joint disorder and/or cardiovascular disease in an individual at risk for such a disorder from occurring.

In embodiments, a method for treating a degenerative joint disorder and/or cardiovascular disease or a degenerative joint disorder- and cardiovascular disease-related complication in a subject is described. The method comprises administering to the subject a composition comprising polycosanols, glucosamine and chondroitin.

As used herein, the term “treatment” and its grammatical equivalents refer to the alleviation or elimination of etiological or pathological symptoms and include, for example, the elimination of such symptom causation either on a temporary or permanent basis, or to alter or slow the appearance of such symptoms or symptom worsening. For example, the term “treatment” includes alleviation or elimination of causation of symptoms associated with, but not limited to, any of the degenerative joint disorders and/or cardiovascular diseases or their related-complications described herein.

As used herein, “therapeutically effective amount” refers to an amount of an active agent that is nontoxic but sufficient to provide the desired effect. The therapeutically effective amount varies according to the patient's sex, age and weight, the route of administration, the nature of the condition and any treatments which may be associated therewith, or any concurrent related or unrelated treatments or conditions of the patient. In determining the effective amount or dose, a number of factors are considered by the attending diagnostician, including, but not limited to, the potency and duration of action of the compounds used, the nature and severity of the illness to be treated, as well as the sex, age, weight, general health and individual responsiveness of the patient to be treated, and other relevant circumstances. Therapeutically effective amounts can be determined without undue experimentation by any person skilled in the art or by following the exemplary guidelines set forth in this application.

Therapeutically effective refers qualitatively to the amount of an agent or agents in combination for use in a degenerative joint disorder and/or cardiovascular disease therapy that will achieve the goal of preventing, or improvement in the severity of, the degenerative joint disorder and/or cardiovascular disease being treated, while avoiding adverse side effects typically associated with alternative therapies. A degenerative joint disorder and/or cardiovascular disease or their related complication symptom is considered ameliorated or improved if any benefit is achieved, irrespective of the absolute magnitude of the amelioration or improvement. For example, any reduction in pain of a subject suffering from a degenerative joint disorder would be considered an ameliorated symptom. Likewise, any inhibition or suppression of vasculature platelets would also be considered amelioration of a cardiovascular disease. Furthermore, any reduction in symptom severity of a degenerative joint disorder and/or a cardiovascular disease or their related complications is considered an ameliorated symptom.

As used herein, the term “subject” for purposes of treatment includes any subject, and preferably is a subject who is in need of the treatment of a degenerative joint disorder and/or a cardiovascular disease, or who needs treatment of a degenerative joint disorder and/or a cardiovascular disease related complication. For purposes of prevention, the subject is any subject, and preferably is a subject that is at risk for, or is predisposed to, developing a degenerative joint disorder and/or cardiovascular disease or their related complication. The subject is typically an animal, more typically is a mammal. Preferably, the mammal is a human, horse, dog or cat.

As used herein, the terms “predisposed to a degenerative joint disorder and/or cardiovascular disease or their related complication” and “at risk for a degenerative joint disorder and/or cardiovascular disorder or their related complication,” both of which are used interchangeably herein, mean any subject at risk for developing degenerative joint disorder and/or cardiovascular disease or any of their related complications. The subject may be at risk due to genetic predisposition, diet, age, exposure to a potentially traumatic environment, exposure to a degenerative joint disorder- and/or cardiovascular disease-causing agents, and the like. The subject may also be at risk due to physiological factors such as anatomical and biochemical abnormalities in the joints and/or heart or vasculature system. For example, obese and/or diabetic subjects are considered at risk for developing a degenerative joint disorder and/or cardiovascular disease as compared to non-obese or non-diabetic subjects.

As used herein, the terms “subject is in need of the prevention or treatment of a degenerative joint disorder and/or a cardiovascular disease related complication” refer to any subject who is suffering from or is predisposed to a degenerative joint disorder and/or cardiovascular disease or any of their related complications described herein. The terms “subject is in need of the prevention or treatment of a degenerative joint disorder and/or cardiovascular disease or their related complications” also refer to any subject that requires a lower dose of therapeutic agents. In addition, the terms “subject is in need of the prevention or treatment of a degenerative joint disorder and/or cardiovascular disease or their related complications” refer to any subject who requires a reduction in the side-effects of a therapeutic agent. Furthermore, the terms “subject is in need of the prevention or treatment of a degenerative joint disorder and/or a cardiovascular disease or their related complications” refer to any subject who requires improved tolerability to any therapeutic agent for degenerative joint disorder and/or cardiovascular disease therapy.

The administration of the components of the herein described formulations may also act synergistically or collectively to provide therapeutic benefits. By way of example, such benefits include: providing sufficient sources of necessary metabolic precursors for the repair and maintenance of connective tissues, providing the proper absorption of these metabolic precursors in the digestive tract, to diminish the inflammatory response in the affected area so that the connective tissue degradation process is halted and/or repair is initiated; suppressing the autoimmune response and any further degradation of tissue in the affected area; stimulating the blood circulatory system, which simultaneously enhances the delivery of the metabolic precursors to the affected areas and removes deleterious deposits in the affected areas; and/or improving lipoprotein profiles and/or reducing fatty acids in the heart muscle.

The method of preventing or treating a degenerative joint disorder and/or a cardiovascular disease or their related complications in a subject that is in need of such prevention or treatment may comprise administering to the subject a composition comprising a polycosanols, chondroitin and glucosamine and optionally at least one additional therapeutic agent as described above. The method may comprise administering a composition comprising polycosanols, chondroitin and glucosamine and optionally PABA and/or folic acid. The composition comprising polycosanols, glucosamine and chondroitin may be administered to a subject in need of such prevention or treatment according to standard routes of oral drug delivery that are well known to one of ordinary skill in the art.

Thus, the herein described method comprises preventing and treating a degenerative joint disorder and/or a cardiovascular disease and their related complication in a subject in need of such prevention and treatment. The method comprises administering an amount of polycosanols, chondroitin and glucosamine with or without PABA and/or folic acid wherein the amount of the polycosanols, chondroitin and glucosamine, and optionally the amount of the PABA and/or folic acid comprises a therapeutically effective amount.

The herein described method comprises preventing and treating a degenerative joint disorder and/or a cardiovascular disease and their related complication in a subject in need of such prevention and treatment may provide for minimizing or preventing degradation of a joint and/or the cardiovascular system of the subject.

In embodiments, the composition of polycosanols, glucosamine and chondroitin may be co-formulated or administered with one or more additional therapeutic agents. Additional therapeutic agents may include for example, analgesics, anesthetics, anti-inflammatories (e.g., non-steroidal anti-inflammatory drugs or corticosteroids) and vitamins.

Exemplary analgesics include procaine, lidocaine, tetracaine, dibucaine, benzocaine, p-butylaminobenzoic acid 2-(diethyl amino) ethyl ester HC1, mepivacaine, piperocaine, dyclonine, morphine, codeine, hydrocodone, and oxycodone.

Examples of useful anesthetics include benzocaine, codeine, dibucaine, dyclonine, hydrocodone, lidocaine, mepivacaine, morphine, oxycodone, p-butylaminobenzoic acid 2-(diethylamino) ethyl ester HCl, piperocaine, procaine and tetracaine.

Any suitable anti-inflammatory agent (e.g., non-steroidal anti-inflammatory drugs, NSAIDs) may be co-formulated with the composition herein described or administered to the mammal being treated with this composition at concentrations known to be effective for these agents. Anti-inflammatory agents include, for example, acetaminophen, aspirin, auranofin, diclofenac, diflunisal, ibuprofen, indomethacin, ketoprofen, naproxen, paracetamol, phenylbutazone, sulindac, Celecoxib, Rofecoxib and steroidal anti-inflammatory agents. Exemplary steroidal anti-inflammatory agents are the corticosteroids such as alclometasone, amcinonide, betamethasone, betamethasone, betamethasone valerate, clobetasol, clocortolone, cortisol, cortisone, desonide, desoximetasone, dexamethasone, diflorasone, flumethasone, fluocinolone acetonide, fluocinonide, fluorometholone, fluprednisolone, flurandrenolide, flurandrenolone acetonide, fluticasone, halcinonide, halobetasol, hydrocortisone, methylprednisolone, mometasone, prednicarbate, prednisolone, prednisone and triamcinolone, and mixtures thereof.

Vitamins include, for example, vitamin B₁₂, vitamin B₆ and combinations thereof. Vitamins also include therapeutically effective derivatives of vitamin B₁₂, and vitamin B₆.

Any therapeutic agent that is typically used in the treatment, prevention, and reduction of degenerative joint disorders may also be administered or co-formulated with the composition herein disclosed. The additional therapeutic agents may be administered within (either before or after) 14 days, 7 days, 24 hours, 12 hours, 1 hour, or simultaneously with the composition and/or formulations herein disclosed.

The composition with or without the additional agents may be provided in a pharmaceutically acceptable carrier or excipient to form a pharmaceutical composition. Pharmaceutically acceptable carriers and excipients include, but are not limited synthetically derived or isolated from a natural source, such as, e.g., hydroxypropyl cellulose; hydroxypropylmethyl cellulose; hydroxyethyl cellulose; methyl cellulose; cellulose acetate; or other cellulose based water soluble polymers and other carriers known in the art. Pharmaceutically acceptable carriers and additives are chosen such that side effects from the pharmaceutical compound are minimized and the performance of the compound is not canceled or inhibited to such an extent that treatment is ineffective.

The composition with or without the additional agents may be administered orally or parenterally by injection, although other effective administration forms, such as intraarticular injection, inhalant mists, transdermal iontophoresis or suppositories are also envisioned. In embodiments, it is envisioned that the carrier and composition constitute a physiologically-compatible, slow release formulation. The carrier may contain other pharmacologically-acceptable excipients for modifying or maintaining the pH, osmolarity, viscosity, clarity, color, sterility, stability, rate of dissolution, taste or odor of the formulation. The carrier may contain still other pharmacologically-acceptable excipients for modifying or maintaining the stability, rate of dissolution, release or absorption of one or more compounds of the composition. Such excipients are those substances usually and customarily employed to formulate dosages for oral or buccal administration in either unit dose or multi-dose form. The compositions herein described are most generally preferably used as tablets, capsules or gelcaps.

The compositions herein disclosed may be formulated as pharmaceutical formulations such that they are suitable for ingestion and may include any form, for example, a pill, capsule, tablet, emulsion, solution, suspension, syrup, buccal or soft gelatin capsule. The pharmaceutical formulations may be designed to provide either immediate or controlled release of any of the components of the formulation. The selection of immediate or controlled release compositions depends upon a variety of factors including the severity of the joint degeneration or cardiovascular disease state. Methods well known in the art for making formulations are found, for example, in Remington The Science and Practice of Pharmacy (20th ed.), ed. A. R. Gennaro, 2000, Lippincott Williams & Wilkins, Philidelphia, or in Encyclopedia of Pharmaceutical Technology, eds. J. Swarbrick and J. C. Boylan, 1988-1999, Marcel Dekker, New York, the pertinent sections being disclosed herein by reference.

All of the components of the composition and/or therapeutic formulations herein disclosed may independently be used within the dose ranges currently known and used for these agents. Each substance may independently be used in excess of the dose ranges currently known and used for these agents. Each substance may independently be used in dose ranges below currently known and used dose ranges for these agents.

Different concentrations of either each substance of the composition or formulation herein described, or the other additional agents may be employed depending on the clinical condition of the patient, the site of joint degeneration, and the severity of the damage. Additional considerations in dose selection include disease etiology, patient age (pediatric, adult, geriatric), general health and comorbidity.

The aforementioned agents may be supplied as pure compounds, or in a form of a pharmaceutically active salt, isomer, a racemic mixture, or in any other chemical form or combination that, under physiological conditions, provides for therapeutically effective treatment of degenerative joint disorders and/or cardiovascular diseases.

The compounds and pharmaceutical formulations described herein may be used with other methods of treating and/or preventing degenerative joint disorders and/or cardiovascular disease. Other methods of treating and/or preventing degenerative joint disorders and/or cardiovascular disease include, for example, a condition for which a cholesterol absorption inhibitor is indicated; preventing or treating a cholesterol related disease; inhibiting the absorption of or reducing plasma or tissue concentration of one or more sterols or stanols; preventing or treating sistoserolemia; dyslipidemia, mixed dyslipidemia, hypo a-lipoproteinemia, LDL pattern B, LDL, pattern A, primary dysbetalipoproteinemia (Frederickson Type ifi), hyperlipidemia (including but not limited to hypercholesterolemia, hypertriglyceridemia, sitosterolemia), hypertension, angina pectoris, reducing blood plasma or serum concentrations of C-reactive protein; preventing, treating, or ameliorating symptoms of Alzheimer's Disease (AD); regulating production or levels of at least one amyloid 3 (Af3) peptide; regulating the amount of ApoE isoform 4 in the bloodstream and/or brain; preventing or treating cognitive related disorders (including dementia); preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cholesterol associated tumors; inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; modulating lipid raft structure; and preventing or treating osteopenia disorders (bone loss disorders). The methods comprise administering a therapeutically effective amount of a compound or pharmaceutical formulation described herein.

By way of example, compounds and pharmaceutical formulations described herein may be used with methods for treating degenerative joint disorders and/or cardiovascular disease together with methods for treating and preventing lipid disorders, such as hypercholesterolemia and hyperlipidemia. Thus, the compounds and formulations described herein may be used advantageously in combination with hypolipidemic agents, including inhibitors of cholesterol biosynthesis, such as HMG-CoA reductase inhibitors. HMG-CoA reductase inhibitors include, for example, the “statins”: lovastatin (Mevacor®), simvastatin (Zocor®), pravastatin (Pravachol®), rosuvastatin (Crestor®; ZD-4522), mevastatin, atorvastatin (Lipitor®)), cerivastatin (Baycol®), pitavastatin, fluvastatin (Lescol®), bervastatin, crilvastatin, carvastatin, rivastatin, sirrivastatin, glenvastatin, itavastatin, dalvastatin as well as those disclosed in U.S. Pat. Nos. 4,231,938, 4,444,784, 4,739,073, 4,346, 227, 4,647,576; and EP 491,226.

The following examples describe embodiments of the invention. It will be appreciated that the amount of the polycosanols, chondroitin and glucosamine with or without additional agents required for use in the treatment or prevention of a degenerative joint disorder and/or a cardiovascular disease and their related complications will vary within wide limits and may be adjusted to the individual requirements of a particular subject. The daily dosage can be administered as a single dosage or in equally or unequally divided dosages.

In general, for administration to adults, an appropriate daily dosage is described herein, although the limits that are identified as being preferred may be exceeded if desired. The dosage level of the amount of any of the polycosanols, chondroitin and glucosamine will necessarily depend on the particular subject.

The following prophetic examples are intended to illustrate the principle of the present invention and circumstances when the formulations herein disclosed are indicated. The following examples are not intended to be limiting.

### PROPHETIC EXAMPLE 1

A formulation suitable for oral or buccal administration to a subject may comprise between 20 milligrams and 100 milligrams polycosanols, 250 milligrams chondroitin and 500 milligrams glucosamine. The formulation may be prepared as one or more of a tablet, capsule or gel-cap suitable for oral administration.

### PROPHETIC EXAMPLE 2

A formulation suitable for oral or buccal administration to a subject may comprise between 10 milligrams and 50 milligrams polycosanols, 125 milligrams chondroitin, 250 milligrams glucosamine and 500 milligrams PABA. The formulation may be prepared as one or more of a tablet, capsule or gel-cap suitable for administration two or more times per day.

### PROPHETIC EXAMPLE 3

A formulation suitable for oral or buccal administration to a subject may comprise between 10 milligrams and 50 milligrams polycosanols, 125 milligrams chondroitin, 250 milligrams glucosamine, 500 milligrams PABA and 0.05 milligrams folic acid. The formulation may be prepared as one or more of a tablet, capsule or gel-cap suitable for oral or buccal administration two or more times per day.

### PROPHETIC EXAMPLE 4

A formulation suitable for oral or buccal administration to a subject may comprise between 20 milligrams and 100 milligrams polycosanols, 250 milligrams chondroitin, 500 milligrams glucosamine and 0.1 milligrams folic acid. The formulation may be prepared as one or more of a tablet, capsule or gel-cap suitable for oral or buccal administration.

### PROPHETIC EXAMPLE 5

A formulation suitable for oral or buccal administration to a subject may comprise between 20 milligrams and 100 milligrams polycosanols, 250 milligrams chondroitin, 500 milligrams glucosamine and 1000 milligrams PABA. The formulation may be prepared as one or more of a tablet, capsule or gel-cap suitable for oral or buccal administration.

### PROPHETIC EXAMPLE 6

A formulation suitable for oral or buccal administration to a subject may comprise between 20 milligrams and 100 milligrams polycosanols, 250 milligrams chondroitin, 500 milligrams glucosamine, 0.1 milligrams folic acid and 1000 milligrams PABA. The formulation may be prepared as one or more of a tablet, capsule or gel-cap suitable for oral or buccal administration.

### PROPHETIC EXAMPLE 7

### Treatment of a Patient Suffering From Rheumatoid Arthritis in an Articular Region

A patient suffering from rheumatoid arthritis in the hips may be treated twice a day, every day, with a composition comprising between 10 milligrams and 50 milligrams polycosanols, 125 milligrams chondroitin, 250 milligrams glucosamine and 500 milligrams PABA. If desired, the patient may also take ibuprofen to reduce pain to the joints.

### PROPHETIC EXAMPLE 8

### Treatment of Osteoarthritis

A geriatric patient diagnosed with osteoarthritis may be administered twice a day, everyday, with a composition comprising between 20 milligrams and 100 milligrams polycosanols, 250 milligrams chondroitin and 500 milligrams glucosamine. The patient may also be injected with cortisone into a particular joint to alleviate the pain.

### PROPHETIC EXAMPLE 9

### Treatment of Cardiovascular Disease

A patient suffering from cardiovascular disease as indicated by elevated lipid levels and/or high blood pressure may be treated twice a day, every day, with a composition comprising between 10 milligrams and 50 milligrams polycosanols, 125 milligrams chondroitin, 250 milligrams glucosamine, 500 milligrams PABA and 0.05 milligrams folic acid. If desired, the patient may also take statins to reduce the lipid levels.

The herein described method comprises preventing and treating a degenerative joint disorder and/or a cardiovascular disease and their related complications in a subject in need of such prevention and treatment by administration of a composition comprising polycosanol, chondroitin and glucosamine to the subject. Methods of diagnosing and monitoring the presence or change of a degenerative joint disorder and/or a cardiovascular disease are generally known. To assess whether the formulations disclosed herein are useful to treat, reduce, or prevent a degenerative joint or cardiovascular disorder, any method known in the art may be used. For example, a medically desirable result for degenerative joint disorder may be a reduction of pain (measured, e.g., using a visual analog pain scale, described for example, by Peyron et al. J. Rheumatol. 20 (suppl.) 39: 10-15 (1993); and/or increased joint mobility (measured, e.g., using pedometry as described in Belcher et al. J. Orthop. Trauma 11:106-109 (1997). Degenerative joint disorders may be diagnosed, for example, by physical examination, by the detection of inflammation in the synovial joints, or by the detection of molecular markers characteristic of such disorders in a biological sample collected from the subject, such as synovial fluid, blood, serum, or urine. By way of example, the diagnosis of any of the cardiovascular disease may be performed by monitoring lipoprotein particle profile (cholesterol subtypes), fibrinogen and PAI-1 blood concentrations, homocysteine level, asymmetric dimethylarginine and/or blood pressure.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.”

As used herein, “consisting of” and grammatical equivalents thereof exclude any element, step, or ingredient not specified in the claim.

As used herein, “consisting essentially of” and grammatical equivalents thereof limit the scope of a claim to the specified materials or steps and those that do not materially affect the basic and novel characteristic or characteristics of the claimed invention.

Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims.

What is claimed is:

1. A therapeutic composition comprising:
a) polycosanols;
b) chondroitin;
c) glucosamine; and
d) optionally PABA, folic acid or combinations thereof;
wherein the polycosanols comprise at least two fatty alcohols selected from tetracosanol, hexacosanol, octacosanol and triacontanol.

2. The therapeutic composition of claim 1, wherein the amount of polycosanols is between 1 milligram and 100 milligrams.

3. The therapeutic composition of claim 1, wherein the amount of chondroitin is between 10 milligrams and 2500 milligrams.

4. The therapeutic composition of claim 1, wherein the amount of glucosamine is between 10 milligrams and 2500 milligrams.

5. The therapeutic composition of claim 1, wherein the wherein the amount of PABA is between 10 milligrams and 2500 milligrams.

6. The therapeutic composition of claim 1, wherein the wherein the amount of folic acid is between 0.005 milligrams and 5 milligrams.

7. The therapeutic composition of claim 1, wherein:
the amount of polycosanols is between 10 milligram and 50 milligrams;
the amount of chondroitin is between 500 milligrams and 2500 milligrams; and
the amount of glucosamine is between 500 milligrams and 2500 milligrams.

8. The therapeutic composition of claim 7, wherein the amount of PABA is between 500 milligrams and 2500 milligrams.

9. The therapeutic composition of claim 7, wherein the amount of folic acid is between 0.01 milligrams and 1 milligram.

10. The therapeutic composition of claim 7, wherein the amount of PABA is between 500 milligrams and 2500 milligrams and the amount of folic acid is between 0.01 milligrams and 1 milligrams.

11. A therapeutic composition consisting essentially of:
a) polycosanols;
b) chondroitin;
c) glucosamine; and
d) optionally PABA, folic acid or combinations thereof;
wherein the polycosanols comprise at least two fatty alcohols selected from tetracosanol, hexacosanol, octacosanol and triacontanol.

12. The therapeutic composition of claim 11, wherein:
the amount of polycosanols is between 10 milligram and 50 milligrams;
the amount of chondroitin is between 500 milligrams and 2500 milligrams; and
the amount of glucosamine is between 500 milligrams and 2500 milligrams.

13. The therapeutic composition of claim 12, wherein the amount of PABA is between 500 milligrams and 2500 milligrams.

14. The therapeutic composition of claim 12, wherein the amount of folic acid is between 0.01 milligrams and 1 milligram.

15. The therapeutic composition of claim 12, wherein the amount of PABA is between 500 milligrams and 2500 milligrams and the amount of folic acid is between 0.01 milligrams and 1 milligrams.

16. The therapeutic composition of claim 1, further comprising an anti-inflammatory agent.

17. The therapeutic composition of claim 12, wherein the anti-inflammatory agent is a NSAID.

18. The therapeutic composition of claim 1, further comprising a carrier for oral or buccal administration.

19. A composition for treating and/or reducing a risk of a cardiovascular disease in a subject in need thereof, the composition comprising:
(a) polycosanols;
(b) at least about 10 milligrams of chondroitin per milligram of the polycosanols;
(c) at least about 10 milligrams of glucosamine per milligram polycosanols;
(d) optionally at least about 10 milligrams of PABA per milligram of the polycosanols; and
(e) optionally at least about 0.005 milligrams of folic acid per milligram polycosanols.

20. A method for treating and/or reducing a risk of a cardiovascular disease in a subject in need thereof, the method comprising:
(A) providing a formulation comprising:
(i) polycosanols;
(ii) at least about 10 milligrams of chondroitin per milligram of the polycosanols;
(iii) at least about 10 milligrams of glucosamine per milligram polycosanols;
(iv) optionally at least about 10 milligrams of PABA per milligram of the polycosanols; and
(v) optionally at least about 0.005 milligrams of folic acid per milligram polycosanols; and
(B) orally administering to the subject for a therapeutically effective period of time a therapeutically effective amount of the formulation.

21. A composition for treating and/or reducing a risk of a degenerative joint disorder in a subject in need thereof, the composition comprising:
a) polycosanols, wherein the polycosanols comprise at least two fatty alcohols selected from tetracosanol, hexacosanol, octacosanol and triacontanol;
(b) at least about 10 milligrams of chondroitin per milligram of the polycosanols;
(c) at least about 10 milligrams of glucosamine per milligram polycosanols;
(d) optionally at least about 10 milligrams of PABA per milligram of the polycosanols; and
(e) optionally at least about 0.005 milligrams of folic acid per milligram polycosanols.

22. A method for treating and/or reducing a risk of a degenerative joint disorder in a subject in need thereof, the method comprising
(A) providing a formulation comprising:
(i) polycosanols, wherein the polycosanols comprise at least two fatty alcohols selected from tetracosanol, hexacosanol, octacosanol and triacontanol;
(ii) at least about 10 milligrams of chondroitin per milligram of the polycosanols;
(iii) at least about 10 milligrams of glucosamine per milligram polycosanols;
(iv) optionally at least about 10 milligrams of PABA per milligram of the polycosanols; and
(v) optionally at least about 0.005 milligrams of folic acid per milligram polycosanols; and
(B) orally administering to the subject for a therapeutically effective period of time a therapeutically effective amount of the formulation.
